SEQUENCE LISTING (2 sequences)

SEQ ID NO: 1 - protein, 230 aa, synthetic construct
  REGION 1..230: note = Description of sequence: Sequence A (Aspartic Acid Racemase)
MKTIGLLGGMSWESTIPYYRLINEGIKQRLGGLHSAQVLLHSVDFHEIEECQRRGEWDKTGDILAEAALGLQRAGAEGIVLCTNTMHKVADAIESRCTLPFLHIADATGRAITGAGMTRVALLGTRYTMEQDFYRGRLTEQFSINCLIPEADERAKINQIIFEELCLGQFTEASRAYYAQVIARLAEQGAQGVIFGCTEIGLLVPEERSVLPVFDTAAIHAEDAVAFMLS

SEQ ID NO: 2 - protein, 230 aa, synthetic construct
  REGION 1..230: note = Description of sequence: Sequence B (Asparagine Racemase)
MKTIGLLGGMSWESTIPYYRLINEGIKQRLGGLHSAQVLLHSVDFHEIEECERRGEWDKTGDILAEAALGLQRAGAEGIVLCTNTMHKVADAIESRCTLPFLHIADATGRAITGAGMTRVALLGTRYTMEQDFYRGRLTEQFSINCLIPEADERAKINQIIFEELCLGQFTEASRAYYAQVIARLAEQGAQGVIFGCTEIGLLVPEERSVLPVFDTAAIHAEDAVAFMLS

(19) United States
(12) Patent Application Publication
(10) Pub. No.: US 2025/0144189 A1
(43) Pub. Date: May 8, 2025

(54) ASPARAGINE RACEMASE AND USES THEREOF

(71) Applicant: BIOXEL, INC., Cambridge, MA (US)

(72) Inventors: Patrick R. GLEASON, Millis, MA (US); Elazer R. EDELMAN, Brookline, MA (US)

(73) Assignee: BIOXEL, INC., Cambridge, MA (US)

(21) Appl. No.: 18/936,118

(22) Filed: Nov. 4, 2024

Related U.S. Application Data
(60) Provisional application No. 63/596,559, filed on Nov. 6, 2023.

(51) Int. Cl. A61K 38/52; C12N 9/90

(52) U.S. Cl. CPC A61K 38/52; C12N 9/90; C12Y 501/01003

(57) ABSTRACT

The present disclosure provides a novel recombinant unidirectional asparagine racemase for the conversion of L-asparagine to D-asparagine. Such unidirectional asparagine racemase could be used in treating cancers (e.g., leukemias or lymphomas) that require exogenous L-asparagine. This innovative design offers a promising new approach to reduce detrimental side effects caused by current therapies.

### CROSS-REFERENCE TO RELATED APPLICATIONS

This application claims the benefit of U.S. Provisional Application No. 63/596,559, filed Nov. 6, 2023, which is hereby incorporated by reference.

### SEQUENCE LISTING STATEMENT

A Sequence Listing conforming to the rules of WIPO Standard ST.26 is hereby incorporated by reference. The Sequence Listing has been filed as an electronic document encoded as XML in UTF-8 text. The electronic document, created on Nov. 6, 2023, is entitled “P-629717-USP_ST26.xml”, and is 3,754 bytes in size.

### FIELD OF THE INVENTION

The present disclosure relates in general to the field of cancer treatment. In one embodiment, the present disclosure provides a novel unidirectional asparagine racemase enzyme for treating cancer.

### BACKGROUND OF THE INVENTION

According to the American Cancer Society, there are almost 17 million people living with cancer in the United States. Cancer itself is a broad term used to collect several diseases that share certain characteristics under one heading. Even still, while we label cancers that affect a specific tissue as a singular group in everyday language (e.g., lung cancer for all cancers that affect the lungs), there are actually numerous genotypes present throughout the population. This high variability in turn requires a large number of diverse treatment options.

One particular type of treatment, known as “enzyme therapy”, utilizes enzymes to break down small molecules that either are obligate metabolites of cancer cells or that act as immunosuppressants that inhibit the body's own cancer defense mechanisms. An example is the use of L-Asparaginase (ASNase) to treat acute lymphocytic leukemia (ALL). L-asparaginase has also been used to treat Hodgkin's disease, acute myelocytic leukemia, acute myelomonocytic leukemia, chronic lymphocytic leukemia, lymphosarcoma, reticulosarcoma, and melanosarcoma.

While normal, healthy cells are able to synthesize the amino acid L-asparagine (L-ASN) for use in protein synthesis, certain malignant cells cannot; meaning that those malignant cells must obtain L-ASN from circulating blood for survival. By reducing the amount of circulating L-ASN, ASNase effectively starves ALL cells eventually leading to tumor reduction.

Therapies using ASNase are quite effective with survival rates above 70%. Yet, side-effects can and do occur in greater than 30% of patients. The primary reason for this is that ASNase degrades L-ASN into two components: ammonia and L-aspartic acid. Both degradation products can have detrimental side effects on the central nervous systems of patients.

A different, yet comparable, approach may be to convert L-asparagine to the biologically inert D-asparagine (D-ASN). Unlike L-amino acids, D-amino acids cannot be used by cells for protein synthesis, thus accomplishing the same task as ASNase of starving malignant cells of L-ASN. And since this is an isomerization reaction instead of a degradation reaction, harmful byproducts are avoided altogether. Typically, the conversion of an L-amino acid to a D-amino acid and the reverse reaction of D-amino acid to L-amino acid is performed by an enzyme known as a racemase.

Racemases are defined by their ability to non-specifically convert L-amino acids to D-amino acids and maintain a chemical equilibrium between the two isomers. There are two known classes of racemases, those that require the 5-pyridoxal phosphate (PLP-dependent) and those that do not (PLP-independent). Racemases have been found to convert a number of amino acids, such as alanine, serine, arginine, aspartic acid, glutamic acid, and proline to name a few, from one isomer to another. However, no L-ASN racemase with both high specificity and high enzymatic activity for the conversion of L-ASN to D-ASN has been discovered. Instead, several other amino acid racemases (e.g., L-serine racemase) show weak specificity for L-ASN.

Hence, there continues to be a need for developing a unidirectional asparagine racemase that would be useful for the treatment of cancer and for improved methods for treating cancer, in particular, Acute Lymphoblastic Leukemia and Non-Hodgkin Lymphoma.

### SUMMARY OF THE INVENTION

In one aspect, the present disclosure provides a recombinant asparagine racemase capable of converting L-asparagine to D-asparagine. In an embodiment of the asparagine racemase, the asparagine racemase is a unidirectional asparagine racemase. In another embodiment of the asparagine racemase, the asparagine racemase is derived from modifying an aspartic or glutamic acid racemase from Escherichia coli. In some embodiments of the asparagine racemase, the modification comprises a mutation of glutamine to glutamic acid at amino acid position 52 (Q52E). In an embodiment of the asparagine racemase, the asparagine racemase retains the active site amino acids of serine 11 (S11), threonine 83 (T83), asparagine 84 (N84), threonine 85 (T85), cysteine 197 (C197), threonine 198 (T198), and glutamic acid 199 (E199). In an embodiment of the asparagine racemase, the asparagine racemase comprises the active site amino acids of serine 11 (S11), threonine 83 (T83), asparagine 84 (N84), threonine 85 (T85), cysteine 197 (C197), threonine 198 (T198), and a mutation of glutamic acid 199 (E199) to aspartic acid (E199D) or histidine (E199H). In another embodiment of the asparagine racemase, the asparagine racemase comprises the amino acid sequence of SEQ ID NO:2.

In another aspect, the present disclosure provides a pharmaceutical composition comprising the herein provided recombinant asparagine racemase capable of converting L-asparagine to D-asparagine and a pharmaceutically acceptable carrier. In an embodiment of the provided pharmaceutical composition, the asparagine racemase is a unidirectional asparagine racemase. In another embodiment of the pharmaceutical composition, the asparagine racemase is derived from modifying an aspartic or glutamic acid racemase from Escherichia coli. In some embodiments of the pharmaceutical composition, the modification of the aspartic or glutamic acid racemase from E. coli comprises a mutation of glutamine to glutamic acid at amino acid position 52 (Q52E). In an embodiment of the pharmaceutical composition, the herein provided asparagine racemase retains the active site amino acids of serine 11 (S11), threonine 83 (T83), asparagine 84 (N84), threonine 85 (T85), cysteine 197 (C197), threonine 198 (T198), and glutamic acid 199 (E199). In an embodiment of the pharmaceutical composition, the herein provided asparagine racemase comprises the active site amino acids of serine 11 (S11), threonine 83 (T83), asparagine 84 (N84), threonine 85 (T85), cysteine 197 (C197), threonine 198 (T198), and a mutation of glutamic acid 199 (E199) to aspartic acid (E199D) or histidine (E199H). In another embodiment of the pharmaceutical composition, the herein provided asparagine racemase comprises the amino acid sequence of SEQ ID NO:2.

In one aspect, the present disclosure provides methods for treating a cancer in a subject in need thereof, comprising administering to said subject a therapeutically effective amount of the recombinant asparagine racemase capable of converting L-asparagine to D-asparagine. In some embodiments of said methods asparagine racemase, the asparagine racemase comprises the amino acid sequence of SEQ ID NO:2. In an embodiment of said methods, the cancer is Acute Lymphocytic Leukemia (ALL). In another embodiment of said methods, the cancer is Non-Hodgkin Lymphoma.

These and other aspects of the invention will be appreciated from the ensuing descriptions of the figures and detailed description of the invention.

### BRIEF DESCRIPTION OF THE DRAWINGS

Some embodiments of the invention are herein described, by way of example only, with reference to the accompanying drawing. With specific reference now to the drawing in detail, it is stressed that the particulars shown are by way of example and for purposes of illustrative discussion of embodiments of the invention. In this regard, the description taken with the drawing makes apparent to those skilled in the art how embodiments of the invention may be practiced.

FIG. 1 depicts one embodiment of an irreversible reaction of L-asparagine to D-asparagine facilitated by the presently described recombinant unidirectional asparagine racemase.

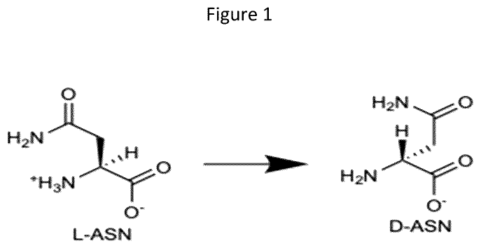

### DETAILED DESCRIPTION OF THE INVENTION

In recent years, a small group of PLP-independent aspartic acid racemase variants have been identified that allow the reaction to move forward in only one direction, from L-aspartic acid to D-aspartic acid. Directionality is facilitated when one of the catalytic cysteines is mutated to either a serine or more preferentially a threonine. This new subclass of enzymes has been termed unidirectional racemases. Given the lack of a catalytically efficient asparagine racemase and the similarities between aspartic acid and asparagine it should appear feasible to engineer a novel enzyme that can accommodate high specificity for the conversion of L-ASN to D-ASN.

In one embodiment, the present disclosure provides a novel unidirectional asparagine racemase designed for efficient conversion of the isomeric amino acid L-Asparagine to D-Asparagine. The present unidirectional asparagine racemase could potentially revolutionize the approach to cancer therapy. In one embodiment, the present unidirectional asparagine racemase offers a therapeutic alternative for patients with various leukemias and lymphomas, particularly Acute Lymphocytic Leukemia (ALL), by substituting traditional asparaginase enzyme treatments, resulting in reduced adverse side-effects and providing a more favorable therapeutic profile. To overcome the lack of naturally occurring asparagine racemases, a novel unidirectional asparagine racemase was designed, starting from a homodimeric unidirectional aspartic/glutamic acid racemase found in certain strains of the bacteria Escherichia coli, e.g., E. coli O157:1-17 (Taxon ID 83334) (UniProt entry A0A0H3JGH6·YGEA_ECO57).

In one embodiment, the aspartic acid aspartic/glutamic acid racemase (also called aspartate/glutamate-specific racemase) has the following amino acid sequence:

(SEQ ID NO: 1)
MKTIGLLGGMSWESTIPYYRLINEGIKQRLGGLHSAQVLLHSVDFHEIEE
CQRRGEWDKTGDILAEAALGLQRAGAEGIVLCTNTMHKVADAIESRCTLP
FLHIADATGRAITGAGMTRVALLGTRYTMEQDFYRGRLTEQFSINCLIPE
ADERAKINQIIFEELCLGQFTEASRAYYAQVIARLAEQGAQGVIFGCTEI
GLLVPEERSVLPVEDTAAIHAEDAVAFMLS.

In one embodiment, the sequence of the aspartic acid racemase was altered to have glutamine 52 mutated to glutamic acid (Q52E) and maintaining the active site amino acids of serine 11 (S11), threonine 83 (T83), asparagine 84 (N84), threonine 85 (T85), cysteine 197 (C197), threonine 198 (T198), and glutamic acid 199 (E199). In another embodiment, the glutamine 52 mutated to glutamic acid Q52D; however, such a mutation may result in loss of function of the active site.

While the C197 and E199 catalytic dyad is responsible for dehydrogenation, T85 is critically important for hydrogenation and inhibition of the reverse reaction of D-ASN to L-ASN. The amino acid residue 199 may be E199, E199D or E199H. The remaining residues 511, T83, N84, and T198 are critical for the orientation of the substrate in the active site.

In one embodiment, the recombinant unidirectional asparagine racemase has the following amino acid sequence:

(SEQ ID NO: 2)
MKTIGLLGGMSWESTIPYYRLINEGIKQRLGGLHSAQVLLHSVDFHEIEE
CERRGEWDKTGDILAEAALGLQRAGAEGIVLCTNTMHKVADAIESRCTLP
FLHIADATGRAITGAGMTRVALLGTRYTMEQDFYRGRLTEQFSINCLIPE
ADERAKINQIIFEELCLGQFTEASRAYYAQVIARLAEQGAQGVIFGCTEI
GLLVPEERSVLPVFDTAAIHAEDAVAFMLS.

As used herein, the terms “comprise”, “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “an enzyme” or “at least one enzyme” may include a plurality of enzymes, including mixtures thereof.

Throughout this application, various embodiments of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable. In some embodiments, the term “about”, refers to a deviance of between 0.0001-5% from the indicated number or range of numbers. In some embodiments, the term “about”, refers to a deviance of between 1-10% from the indicated number or range of numbers. In some embodiments, the term “about”, refers to a deviance of up to 25% from the indicated number or range of numbers. The term “comprises” means encompasses all the elements listed, but may also include additional, unnamed elements, and it may be used interchangeably with the terms “encompasses”, “includes”, or “contains” having all the same qualities and meanings. The term “consisting of” means being composed of the recited elements or steps, and it may be used interchangeably with the terms “composed of” having all the same qualities and meanings. Further, reference to values stated in ranges includes each and every value within that range. Certain features of the disclosed compositions and methods which are described herein in the context of separate aspects may also be provided in combination in a single aspect. Alternatively, various features of the disclosed compositions and methods that are, for brevity, described in the context of a single aspect, may also be provided separately or in any subcombination.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

In one embodiment, there is provided a recombinant asparagine racemase capable of converting L-asparagine to D-asparagine. In one embodiment, the asparagine racemase is a unidirectional asparagine racemase. In one embodiment, the unidirectional asparagine racemase is derived from modifying an aspartic or glutamic acid racemase from Escherichia coli. In one embodiment, the modification comprises a mutation of glutamine to glutamic acid at amino acid position 52 (Q52E). In one embodiment, the asparagine racemase retains the active site amino acids of serine 11 (S11), threonine 83 (T83), asparagine 84 (N84), threonine 85 (T85), cysteine 197 (C197), threonine 198 (T198), and glutamic acid 199 (E199), wherein said active site is the active site of homodimeric unidirectional aspartic/glutamic acid racemase found in certain strains of the bacteria Escherichia coli, as described below. The herein described recombinant unidirectional asparagine racemases are designed around the active site architecture.

In one embodiment, the recombinant unidirectional asparagine racemase comprises the amino acid sequence of SEQ ID NO:2. In some embodiments, the recombinant unidirectional asparagine racemase comprises an amino acid sequence which is 25, 30, 35, 40, 45, 50, 55, 60, 65, 70, 75, 80, 85, 90, 95, or 99% identical to the sequence set forth in SEQ ID NO: 2. In an embodiment, the recombinant unidirectional asparagine racemase comprises an amino acid sequence which is 25% identical to the sequence set forth in SEQ ID NO: 2. In some embodiments, the recombinant unidirectional asparagine racemase comprises an amino acid sequence which is 30% identical to the sequence set forth in SEQ ID NO: 2. In an embodiment, the recombinant unidirectional asparagine racemase comprises an amino acid sequence which is 35% identical to the sequence set forth in SEQ ID NO: 2. In some embodiments, the recombinant unidirectional asparagine racemase comprises an amino acid sequence which is 40% identical to the sequence set forth in SEQ ID NO: 2. In certain embodiments, the recombinant unidirectional asparagine racemase comprises an amino acid sequence which is 45% identical to the sequence set forth in SEQ ID NO: 2. In an embodiment, the recombinant unidirectional asparagine racemase comprises an amino acid sequence which is 50% identical to the sequence set forth in SEQ ID NO: 2. In some embodiments, the recombinant unidirectional asparagine racemase comprises an amino acid sequence which is 55% identical to the sequence set forth in SEQ ID NO: 2. In an embodiment, the recombinant unidirectional asparagine racemase comprises an amino acid sequence which is 60% identical to the sequence set forth in SEQ ID NO: 2. In various embodiments, the recombinant unidirectional asparagine racemase comprises an amino acid sequence which is 65% identical to the sequence set forth in SEQ ID NO: 2. In certain embodiments, the recombinant unidirectional asparagine racemase comprises an amino acid sequence which is 70% identical to the sequence set forth in SEQ ID NO: 2. In an embodiment, the recombinant unidirectional asparagine racemase comprises an amino acid sequence which is 75% identical to the sequence set forth in SEQ ID NO: 2. In some embodiments, the recombinant unidirectional asparagine racemase comprises an amino acid sequence which is 80% identical to the sequence set forth in SEQ ID NO: 2. In an embodiment, the recombinant unidirectional asparagine racemase comprises an amino acid sequence which is 85% identical to the sequence set forth in SEQ ID NO: 2. In certain embodiments, the recombinant unidirectional asparagine racemase comprises an amino acid sequence which is 90% identical to the sequence set forth in SEQ ID NO: 2. In a particular embodiment, the recombinant unidirectional asparagine racemase comprises an amino acid sequence which is 95% identical to the sequence set forth in SEQ ID NO: 2. In another particular embodiment, the recombinant unidirectional asparagine racemase comprises an amino acid sequence which is 99% identical to the sequence set forth in SEQ ID NO: 2.

The terms “identical” or percent “identity,” in the context of two or more polypeptide and amino acid (or nucleic acid) sequences, refer to two or more sequences or subsequences that are the same or have a specified percentage of amino acid residues (or nucleotides) that are the same (i.e., about 60% identity, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” “Substantially identical” sequences also includes sequences that have deletions and/or additions, as well as those that have substitutions, as well as naturally occurring, e.g., polymorphic or allelic variants, and man-made variants. As described below, the preferred algorithms can account for gaps and the like. Preferably, protein sequence identity exists over a region that is at least about 25 amino acids in length, or in a particular embodiment, over a region that is 50-100 amino acids in length, or more particularly, over the length of a protein.

The recombinant unidirectional asparagine racemase also can have variable lengths of amino acid sequences ranging from 10 amino acid residues to 1000 amino acid residues. In an embodiment, the recombinant unidirectional asparagine racemase comprises an amino acid sequence which is about 15-20, 20-25. 25-30, 30-35, 35-40, 40-45, 45-50, 50-55, 55-60, 60-65, 65-70, 70-75, 75-80, 80-85, 85-90, 90-95 or 95-100 amino acid residues in length. In another embodiment, the recombinant unidirectional asparagine racemase comprises an amino acid sequence which is about 100-125, 125-150, 150-155, 155-160, 160-165, 165-170, 170-175, 175-180, 180-185, 185-190, 190-195 or 195-200 amino acid residues in length. In some embodiments, the recombinant unidirectional asparagine racemase comprises an amino acid sequence which is about 200-225, 225-250, 250-255, 255-260, 260-265, 265-270, 270-275, 275-280, 280-285, 285-290, 290-295 or 295-300 amino acid residues in length. In an embodiment, the recombinant unidirectional asparagine racemase comprises an amino acid sequence which is about 300-325, 325-350, 350-355, 355-360, 360-365, 365-370, 370-375, 375-380, 380-385, 385-390, 390-395 or 395-400 amino acid residues in length. In some embodiments, the recombinant unidirectional asparagine racemase comprises an amino acid sequence which is about 400-425, 425-450, 450-455, 455-460, 460-465, 465-470, 470-475, 475-480, 480-485, 485-490, 490-495 or 495-500 amino acid residues in length. In an embodiment, the recombinant unidirectional asparagine racemase comprises an amino acid sequence which is about 500-525, 525-550, 550-555, 555-560, 560-565, 565-570, 570-575, 575-580, 580-585, 585-590, 590-595 or 595-600 amino acid residues in length.

In another embodiment, the recombinant unidirectional asparagine racemase comprises an amino acid sequence which is about 600-625, 625-650, 650-655, 655-660, 660-665, 665-670, 670-675, 675-680, 680-685, 685-690, 690-695 or 695-700 amino acid residues in length. In an embodiment, the recombinant unidirectional asparagine racemase comprises an amino acid sequence which is about 700-725, 725-750, 750-755, 755-760, 760-765, 765-770, 770-775, 775-780, 780-785, 785-790, 790-795 or 795-800 amino acid residues in length. In another embodiment, the recombinant unidirectional asparagine racemase comprises an amino acid sequence which is about 800-825, 825-850, 850-855, 855-860, 860-865, 865-870, 870-875, 875-880, 880-885, 885-890, 890-895 or 895-900 amino acid residues in length. In an embodiment, the recombinant unidirectional asparagine racemase comprises an amino acid sequence which is about 900-925, 925-950, 950-955, 955-960, 960-965, 965-970, 970-975, 975-980, 980-985, 985-990, 990-995 or 995-1000 amino acid residues in length.

The critical feature of the herein provided recombinant unidirectional asparagine racemases is the spatial arrangement of the cysteine (197), threonine (52), and glutamic acid (199) catalytic residues and the glutamic acid (52) responsible for ASN recognition.

The present disclosure also provides a pharmaceutical composition comprising the recombinant unidirectional asparagine racemase disclosed herein and a pharmaceutically acceptable carrier.

As used herein, a “pharmaceutically acceptable carrier” is well known to those skilled in the art. The carrier may be a solid carrier for solid formulations, a liquid carrier or diluent for liquid formulations, or mixtures thereof. In addition, the pharmaceutical compositions of the invention may further include one or more ingredient selected from diluents, buffers, flavoring agents, binders, disintegrants, surface active agents, thickeners, lubricants, preservatives (including antioxidants), and the like. The formulations may be of immediate release, sustained release, delayed-onset release or any other release profile known to one skilled in the art.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the recombinant asparagine racemase disclosed herein, use thereof in the compositions is contemplated. Supplementary active compounds can also be incorporated into the compositions.

Moreover, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

The pharmaceutical compositions may be prepared by methodology well known in the pharmaceutical art. For example, a pharmaceutical composition intended to be administered by injection can be prepared by combining a composition that comprises the recombinant unidirectional asparagine racemase disclosed herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the enzyme composition so as to facilitate dissolution or homogeneous suspension of the enzyme in the aqueous delivery system.

The compositions may be administered in a therapeutically effective amount, which will vary depending upon a variety of factors including the activity of the enzyme employed; the metabolic stability and length of action of the enzyme; the age, body weight, general health, sex, and diet of the patient; the mode and time of administration; the rate of excretion; the drug combination; the severity of the particular disorder or condition; and the subject undergoing therapy. A “therapeutically effective amount” as used herein refers to that amount which provides a therapeutic effect for a given indication and administration regimen.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. In a particular embodiment, the disease is cancer, The cancers which may be treated with the pharmaceutical compositions comprising a recombinant unidirectional asparagine racemase and a pharmaceutically acceptable carrier.

The terms “subject,” “individual,” and “patient” are used interchangeably herein, and refer to an animal, for example a human, to whom treatment for one or more cancer, including prophylactic treatment, with the pharmaceutical compositions according to the present invention, i.e., the herein described recombinant unidirectional asparagine racemase therapeutic agents, respectively, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys.

The present disclosure also provides a method for treating a disease or condition (e.g., cancer) in a subject in need thereof, comprising administering to the subject a therapeutically effective amount of the recombinant asparagine racemase disclosed herein. The present disclosure also provides use of the recombinant asparagine racemase disclosed herein for treating a disease or condition (e.g., cancer) in a subject in need thereof. In one embodiment, the recombinant unidirectional asparagine racemase comprises the amino acid sequence of SEQ ID NO:2. In one embodiment, the cancer is Acute Lymphocytic Leukemia (ALL). In an embodiment, the cancer is Non-Hodgkin Lymphoma.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treat”, “treating” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented.

In one embodiment, the disease is a cancer that can be, but is not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

In one embodiment, the recombinant unidirectional asparagine racemase disclosed herein could be used for treating cancers that require exogenous L-asparagine. Examples of such cancer include, but are not limited to, leukemias or lymphomas. In one embodiment, the cancer is Acute Lymphocytic Leukemia (ALL). In an embodiment, the cancer is Non-Hodgkin Lymphoma.

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the present disclosure may be administered only once, or it may be administered multiple times. For multiple dosages, the composition may be, for example, administered three times a day, twice a day, once a day, once every two days, twice a week, weekly, once every two weeks, or monthly, or any other schedule generally known in the art.

The composition, i.e., therapeutic agent, of the present disclosure may be administered via as described herein and a pharmaceutical composition comprising the same can be administered to a subject by any method known to a person skilled in the art. These administration methods include, but are not limited to, orally, parenterally, intravascularly, paracancerally, transmucosally, transdermally, intramuscularly, intranasally, intravenously, intradermally, subcutaneously, sublingually, intraperitoneally, intraventricularly, intracranially, intravaginally, by inhalation, rectally, or intratumorally. These methods include any means in which the compound or the pharmaceutical composition comprising the same can be delivered to tissue (e.g., needle or catheter).

In the description presented herein, each of the steps of the invention and variations thereof are described. This description is not intended to be limiting and changes in the components, sequence of steps, and other variations would be understood to be within the scope of the present invention.

Various embodiments and aspects of the present invention are delineated hereinabove. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A recombinant asparagine racemase capable of converting L-asparagine to D-asparagine.

2. The asparagine racemase of claim 1, wherein said asparagine racemase is a unidirectional asparagine racemase.

3. The asparagine racemase of claim 1, wherein said asparagine racemase is derived from modifying an aspartic or glutamic acid racemase from Escherichia coli.

4. The asparagine racemase of claim 3, where the modification comprises a mutation of glutamine to glutamic acid at amino acid position 52 (Q52E).

5. The asparagine racemase of claim 4, wherein said asparagine racemase retains the active site amino acids of serine 11 (S11), threonine 83 (T83), asparagine 84 (N84), threonine 85 (T85), cysteine 197 (C197), threonine 198 (T198), and glutamic acid 199 (E199).

6. The asparagine racemase of claim 4, wherein said asparagine racemase comprises the active site amino acids of serine 11 (S11), threonine 83 (T83), asparagine 84 (N84), threonine 85 (T85), cysteine 197 (C197), threonine 198 (T198), and a mutation of glutamic acid 199 (E199) to aspartic acid (E199D) or histidine (E199H).

7. The asparagine racemase of claim 1, where the asparagine racemase comprises the amino acid sequence of SEQ ID NO:2.

8. A pharmaceutical composition comprising the recombinant racemase of claim 1 and a pharmaceutically acceptable carrier.

9. A method for treating a cancer in a subject in need thereof, comprising administering to said subject a therapeutically effective amount of the recombinant asparagine racemase of claim 1.

10. The method of claim 9, wherein said asparagine racemase comprises the amino acid sequence of SEQ ID NO:2.

11. The method of claim 9, wherein the cancer is Acute Lymphocytic Leukemia (ALL).

12. The method of claim 9, wherein the cancer is Non-Hodgkin Lymphoma.
